(19) United States
(12) Patent Application Publication
(10) Pub. No.: US 2003/0008922 A1
(43) Pub. Date: Jan. 9, 2003

(54) Multiple drug resistance reversal agent

(76) Inventors: Merritt B. Andrus, Provo, UT (US); Timothy M. Turner, Provo, UT (US); John H. Prince, Provo, UT (US)

Correspondence Address: MADSON & METCALF, GATEWAY TOWER WEST, SUITE 900, 15 WEST SOUTH TEMPLE, SALT LAKE CITY, UT, 84101

(21) Appl. No.: 10/203,724

(22) PCT Filed: Feb. 16, 2001
(86) PCT No.: PCT/US01/04920

(51) Int. Cl. A61K 31/16

(52) U.S. Cl. 514/616

(57) ABSTRACT

The present invention relates to a MDR reversal agent. The agent is polyvalent possessing two or more binding domains spaced to effectively inhibit the multiple drug resistance activity of Pgp. The MDR reversal agent is based upon the naturally occurring compound (−)-stipiamide. The multiple drug resistence reversal agent of the present invention can be a homodimer based on napthyl-DHS. The homodimer incorporates two napthyl-DHS domains joined by a series of joined ethylene glycol spacers. The invention also relates to method of reversing MDR in a human cell by administering the reversal agent of the invention. When Pgp is contacted with the reversal agent, the ATPase activity of Pgp is significantly reduced as well as the binding affinity of Pgp for its known substrates.

### RELATED APPLICATIONS

This application is related to and claims the benefit of U.S. Provisional Application Serial No. 60/182,900 of Merritt B. Andrus, filed Feb. 16, 2000 and entitled “Dual Domain Homo-dimeric Polyene MDR Reversal Agents” which is incorporated herein by this reference.

### GOVERNMENT RIGHTS

This invention was made with U.S. Government support under Grant No. ROI GM 57275-01 awarded by the National Institutes of Health. The U.S. Government may have certain rights in the invention.

### FIELD OF THE INVENTION

The present invention relates to agents for the treatment of cancer. More specifically, the invention relates to agents which reverse multiple drug resistance in many types of cancer.

### TECHNICAL BACKGROUND

Cancer involves the out-of-control growth and spread of abnormal cells. Normally, human cells grow, divide, and die in a preprogramed manner. When a person is young, most cell types divide more rapidly than at later stages of development. When that person becomes an adult, the rate of cell division slows dramatically. In an adult, normal cells of most tissues divide only to replace worn-out or dying cells and to repair injuries.

However, cancer cells grow and divide in a rapid, abnormal fashion. These rapid growing cells can start in one part of the body and spread to another location. These cells accumulate and form lumps know as tumors. The tumors can compress, invade, and destroy normal tissue. When cells break away from tumors, the cells can travel through the bloodstream or the lymphatioc system to other areas of the body. There, the cancerous cells may settle and form “colony” tumors. In the new location, the cancer cells continue to grow.

It is estimated that in the United States one-half of all men and one-third of all women will develop some form of cancer during their lifetimes. Today, when many types of cancers are detected early enough, the cancer may be cured or controlled. Millions of people are living with cancer or have been cured of the disease.

Cancer is currently treated with a combination of methods. The means of treating the cancer depends on the stage of the tumor and the type of cancer. Treatment options may include surgery, radiation, chemotherapy, hormone therapy, and immunotherapy. Radiation therapy uses high-energy particles or waves, such as x-rays or gamma rays, to destroy or damage cancer cells. Hormone therapy is treatment with hormones, drugs that interfere with hormone production or hormone action, or surgical removal of hormone-producing glands to kill cancer cells or slow their growth. Immunotherapy is the use of treatments that promote or support the body's immune system response to a disease such as cancer.

With localized cancers, surgery is one of the oldest and most frequently used forms of treatment for cancer. Surgery offers the greatest chance for cure for many types of cancer. Approximately 60% of people with cancer will have some type of surgery. Surgery is also frequently used to remove the bulk of a tumor while another method of treatment such as chemotherapy is used to treat any residual cells of the cancer.

For cancers that have spread from one location to another, chemotherapy is the therapy of choice. Chemotherapy uses anti-cancer drugs. Systemic chemotherapy uses anti-cancer drugs given orally or interveinously. The chemotherapy drugs enter the bloodstream and reach all areas of the body, making it possible to treat a spreading cancer.

Multiple drug resistence is a major problem in the treatment of cancer with chemotherapy. For example, leukemia, a form of cancer, does not usually form a tumor. Instead, these cancer cells involve the blood and blood-forming organs (bone marrow, lymphatic system, and spleen), and circulate through other tissues where they can accumulate. Leukemia cells have been isolated that developed multiple drug resistance (MDR) to anti-cancer drugs. Generally, the MDR occurs when the leukemia cells transfer certain chemotherapy drugs from inside their cell to the outside. This prevents the drugs from accumulating inside the cells at levels high enough to kill the cancer cells.

Multiple drug resistance is not only a problem with leukemia, but has been observed in cancer cells of all types of cancer. Multi-drug resistance (MDR) is due primarily to the expression of Pgp (P-glycoprotein), an ATP-driven membrane-bound multi-drug transporter. Pgp has multiple sites which can bind and transport anti-cancer drugs. These multiple sites make Pgp a powerful causal agent in MDR. Nearly all forms of cancer have been shown to develop resistance mainly through the overexpression of Pgp.

While normally present only at very low levels, Pgp is greatly over expressed in drug resistant cancer cells, cells that become cross resistant to multiple chemotherapeutic agents following extended exposure to an anti-cancer agent. Resistant forms are most common with cancers that have reoccurred following previous episodes. Pgp has now been detected in most classes of cancer cells including adrenocortical, bladder, brain, breast, colon, head and neck, liver, Hodgkin's lymphoma, lung, esophagus, ovarian, renal, retinoblastoma, soft tissue sarcoma, stomach, testes, and thyroid. In most instances, Pgp expression is believed to be responsible for the failure of chemotherapy, the clinical manifestation of MDR.

Various transport inhibitors have been investigated as MDR reversal agents. Most have been known compounds approved for other applications, for example, verapamil, the calcium ion channel blocker used to treat hypertension, the immunosuppresant cyclosporin and its analogs, and known kinase inhibitors, such as staurosporin. Many of the agents used to treat MDR must be used at high levels to show effective reversal of the drug resistance. These agents can be expensive which increases the already great expense of cancer treatment. Moreover, the agents can be quite toxic to normal cells.

Recently, a natural product stipiamide has been shown to an effective MDR reversal agent. Stipiamide was discovered using a specific MDR screen targeting Pgp activity. Stipiamide, unlike other MDR reversal agents is not an analog of known channel blockers and not approved for other purposes. Stipiamide has been chemically, synthesized making it possible to make large amounts of it to treat MDR. Andrus, M. B. & Lepore, S. D. J. Am. Chem. Soc. 1997, 119: 2327 However, stipiamid is highly toxic and can harm normal cells. Additionally, stipiamide can only bind one of the potential drug binding sites on Pgp requiring more stipiamide to completely shut down Pgp.

Polyvalency has become a popular strategy for increasing the binding affinity of ligands to multimeric receptors such as Pgp. Mammen, M. et al., Angew. Chem. Int. Ed. 1998, 37: 2754. A weak binding molecule can be converted into a polyvalent tight binding molecule provided the receptor possesses various binding sites or can be dimerized. The origin of the overall enhanced binding can be attributed to the more favorable second and subsequent binding events where much less entropy is lost due to induced proximity. Among the successful examples are the FK506 and cyclosporin dimers of Schreiber, the vancomycin dimers of Whitesides and Griffin, and the polysaccharides of Kiessling, Whitesides, and Fan. See, e.g., Crabtree, G. R. & Schreiber, S. L., Trends Biocheni. Sci. 1996, 21: 418; Jianghong, R. et al. Chem. & Biol. 1999,6:353; Sundram, U. N. et al. J. Am. Chem. Soc. 1996, 118:13107; Kanai, M. et al. J. Am. Chem. Soc. 1997, 119:9931; Mammen, M. et al. Chen Biol 1996,3:757. Critical issues include the nature of the linker and the polymeric support the length and position of attachment of the linker, and compatibility with assays.

Pgp is a particularly challenging polyvalency target. Pgp is an ATP-dependent drug efflux pump whose overexpression confers multidrug resistance to cancer cells. The development of resistance in cancer cells to chemotherapeutic agents has been a major impediment to effective clinical treatments. Ambudkar, S. V. et al. Ann. Rev. Pharrmwol. Toxicol. 1999, 39: 361; Gottesman, M. M. & Pastan, I. Ann. Rev. Biochem. 1993, 62: 385. To date, no polyvalent anti-Pgp agents have been effectively synthesized.

In light of the foregoing, it would be an advancement in the art to provide a MDR reversal agent which can reverse multiple drug resistance caused by Pgp. It would be a further advancement if the agent were less toxic to normal cells than currently available MDR reversal agents. It would be an additional advancement if the agent could shut down Pgp activity at low concentrations. It would be a further advancement to synthesize a polyvalent anti-Pgp agent. Such agents and methods for using the agents are disclosed and claimed herein.

### BRIEF SUMMARY OF THE INVENTION

The present invention relates to a MDR reversal agent. The agent is polyvalent, possessing two or more binding domains spaced to effectively bind the substrate binding domains of Pgp to which anti-cancer drugs can bind and be transported out of a cancer cell. The MDR reversal agent is based upon the naturally occurring compound (−)-stipiamide. Stipiamide is represented by the following chemical structure:

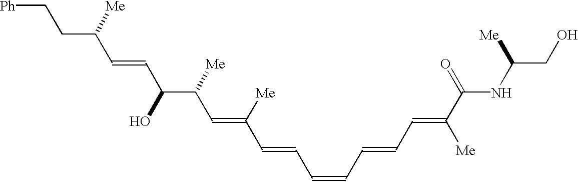

While stipiamide is an effective MDR reversal agent, it is also highly toxic. Recently, 6,7-dehydrostipiamide (DHS) was synthesized. Other MDR agents were also synthesized based on DHS, for example napthyl-DHS is represented by the following chemical structure:

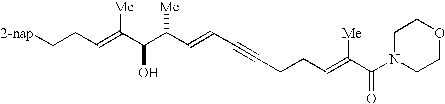

These stipiamide derivatives are less toxic than the naturally occurring stipiamide and are effective at reversing MDR in many types of cancer cells.

The multiple drug resistance reversal agent of the present invention is a polyvalent MDR reversal agent based on stipiamide. The MDR reversal agent has two or more stipiamide-based domains linked into a potent polyvalent MDR reversal agent. The stipiamide-based domains are linked by hydrocarbon linkers. Generally, the domains are spaced by a number of hydrocarbon spacers. The spacers have a length selected to position the nitrogen atoms of the stipiamide-based domains about 3 Å to about 50 Å apart from each other. A polyvalent compound of the present invention may be the homodimer of the following formula:

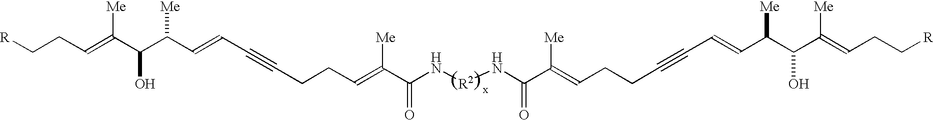

wherein R represents an aromatic group such a benzyl, napthyl, or other substituted phenyl group and R² represents a hydrocarbon spacer such as ethylene glycol and the like. The hydrocarbon spacer can be repeated X times to provide a distance from about 3 Å to about 50 Å between the nitrogen atoms of each of the dimers. A molecule with a distance of about 50 Å between nitrogen atoms has been shown to effectively reverse Pgp activity.

Another embodiment of the MDR reversal agent of the present invention has the following formula.

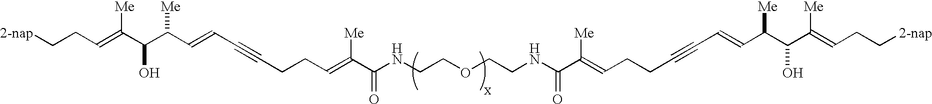

X represents a number of joined ethylene glycol spacers. The number X of joined ethylene glycol spacers can vary between about 0 to about 20 spacers. A group of spacers in the range from about 2 to about 14 spacers can allow for both of the binding domains to tightly bind to Pgp. A length of about 12 spacers has been shown to tightly bind the Pgp binding domains.

The invention also relates to method of reversing multiple drug resistance in a human cell. To reverse MDR in the cancer cell, an effective dose of the MDR reversal agent of the invention is administered to the cell. When the MDR reversal agent is contacted with a Pgp molecule, the ATPase activity of Pgp is significanly decreased suggesting that contacted Pgp will not function as a MDR causing agent. Moreover, the affinity of Pgp for a substrate that is known to bind to Pgp is also reduced in the presence of the MDR agent of the present invention.

These and other advantages of the present invention will become apparent upon reading the following detailed description and appended claims.

### BRIEF DESCRIPTION OF THE DRAWINGS

A more particular description of the invention briefly described above will be rendered by reference to the appended drawings and graphs. These drawings and graphs only provide information concerning typical embodiments of the invention and are not therefore to be considered limiting of its scope.

FIG. 1 depicts chemical structure representations of (−)-stiamide, truncated-DHS, and dual domain stipiamide homodimers.

FIG. 2 depicts a structural representation of a portion of the synthesis of the dual domain homodimers of the present invention.

FIG. 3 is a structural representation of a portion of the synthesis of the dual domain homodimers of the present invention.

FIG. 4 is a structural representation of the synthesis of a monomeric control substrate.

### DETAILED DESCRIPTION OF THE INVENTION

The present invention relates to the synthesis and use of polyvalent MDR reversal with two or more domains based on stipiamide. The domains are configured to prevent substrate binding to Pgp, which has been shown to be the active MDR mechanism in many cancer cell lines. The stipamimde based domains of the polyvalent MDR reversal agents are bound by a series of hydrocarbon spacers such as ethylene glycol. The spacers separate the stipiamide-based domains to allow for optimal binding to Pgp. Generally, such spacers have a length selected to position the nitrogen atoms of the stipiamide-based domains from about 3 Å to about 50 Å apart. In one embodiment of the invention, the MDR reversal agent is a homodimer of the following formula:

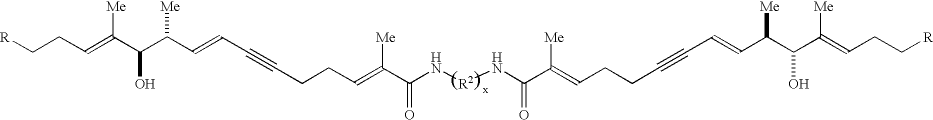

wherein R represents a substituted phenyl group such as napthyl, benzyl, and the like. A number X of hydrocarbon spacers R² join the two domains of the MDR reversal agent. In other configurations, the MDR reversal agent has three or four stipiamide-based domains joined by a tri- or tetra-valent structure. In yet other embodiments, the stipiamide-based domains are joined by a hydrocarbon ring and have five or more stipiamide-based domains.

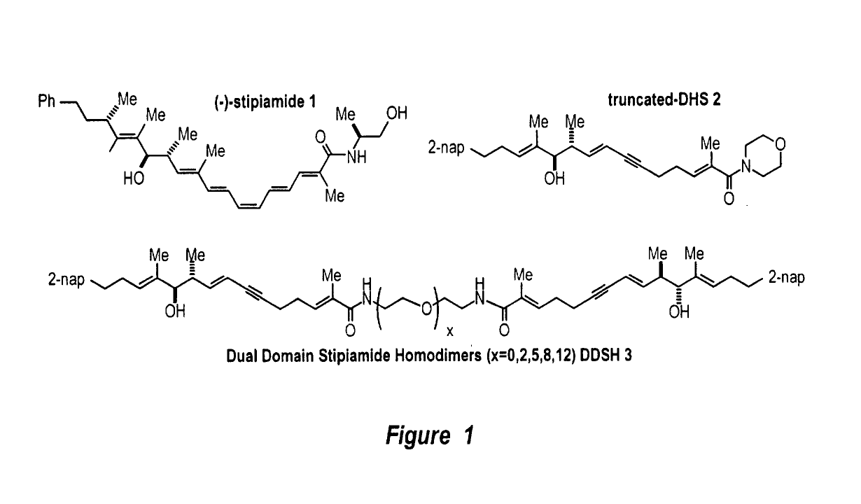

Referring to FIG. 1, a dual-domain MDR reversal agents 3 consists of two stipiamide-based domains bound by polyethylene glycol (PEG) spacers. The MDR agent is designed to span the multiple binding sites on Pgp, occlude the channel, and lead to more effective MDR reversal. PEG spacers of various lengths have been made and attached via a carbamate linkage to two Pgp binding polyenes. The bivalent homodimeric polyene 3 is based on stipiamide 1 and is joined with ethylene glycol spacers. The bivalent homodimer 3 is a strong MDR reversal agent. The homodimer 3 incorporates two of the truncated-DHS monomers 2. The monomers 2 are joined by number of ethylene glycol spacers. This number is shown as X in FIG. 1 and can range from 0 to about 20 ethylene glycol spacers.

Pgp has two binding sites that are associated with its ability to confer multiple drug resistance on a cell. Photoaffinity labeling has been used to identify these two non-identical sites most likely formed by the TM5-6 and 11-12 regions of Pgp. Germann, U. A. Eur. J. Cancer 1996, 32A: 927; Greenberger, L. M. J. Biol. Chem. 1993, 268: 11417; Ramachandra, M. et al Biochemistry 1998, 37: 5010; Dey, S. et al. Proc. Natl. Acad. Sci. 1997, 94: 10594; Sauna, Z. E. & Ambudkar, S. V. Proc. Natl. Acad. Sci. 2000, 97: 2515. The TM5-6 and 11-12 regions are located in close proximity of the cytosolic ATP-binding sites of Pgp. This proximity supports the notion that ATP hydrolysis induces conformational changes that are conveyed to the TM regions leading to drug displacement and efflux. Conformational changes in the substraie-binding domain following ATP hydrolysis have recently been determined. Ramachandra, M. et al., supra; Dey, S. et al., supra; Sauna, Z. E. & Ambudkar, S. V., supra. Additionally, a low resolution (25 Å) electron diffraction structure for Pgp shows the protein to be doughnut shaped with a 50 Å pore opening. Rosenberg, M. F. et al. J. Biol. Chem. 1997, 272: 10685.

The proximity of the TM5-6 and TM 11-12 helices and the identity of residues involved in substrate binding are unknown. However, success of the polyvalency approach to inhibiting Pgp activity requires a proper linker length in order to span the binding sites. A new approach involving displacement of a dimesylate is used to access PEG linkers that now provide for a distance range from 3 to 50 Å. Double Sonogashira couplings are also used to generate the homodimers 3.

The dual-domain compounds 3 are designed by linking two Pgp binding agents with polyene ethylene glycol (PEG) tethers of different lengths. The PEG tethers provide a measure of the distances between proposed intra- and inter-transmembrane binding sites of Pgp. The induced proximity of the two domains increases the effective concentration of the second binding ε vent to Pgp, provided the proper distance is bridged between the two sites. The strategy is inspired by the many examples of induced proximity displayed in cellular processes, ir. particular cell signaling, where receptor dimerization leads to autophosphorylation, and the two-sided peptide antigen interaction between the extracellular T-cell receptor and the major histocompatibility complex of the immune system. Great success has also been found with dimeric FK506, and cyclosporin A ligands. In these examples, the initial binding event greatly enhances the second binding event leading to more favorable complex formation. The effect of induced proximity can now be used to establish the proximity of the proposed TM6 and TM12 binding sites.

The synthetic method uses the optimized Sonogashira coupling conditions developed for the synthesis of the stipiamide analogs. An enyne acid is converted to the acid chloride and reacted with the diamino polyethylene glycol (x=0-12) to form the bis amide in high yields. Sonogashira couplings are then employed with the indicated vinyl iodide to generate the homodimeric Pgp dual domain compounds shown. The napthyl aryl group (Np) is more potent at MDR reversal and is used in the dual-domain compounds. A route to the vinyl iodide uses Masamune's anti-selective norephedrine aldol reaction to set the anti-1,2-hydroxy methyl stereochemistry. The sequence has been applied to diamine linkers of various sizes ranging from x=0 to 12.

The dual-domain homo-dimeric compounds are made using a N,N′-disuccinimidyl carbonate (DSC) based strategy for the formation of carbamate linked material. DSC is used to couple hindered alcohols with highly functionalized amines at 1:1 stoichiometry in a single-flask operation giving carbamates in high yield. Mono-protected (TBS-) tri-, hepta-, and dodeca-ethylene glycols are reacted with N,N′-disuccinimidyl carbonate (DSC) and triethylamine in acetonitrile to give the bis-succinimidyl carbonate intermediates. Addition of the amine amide shown then produces the protected PEG carbamate products. Sonogashira coupling with the vinyl iodide also occurrs in good yield. Removal of the TBS ether gives the alcohol that is again subjected to DSC coupling. The amine now is doxorubricin (adriamycin), the potent anti-cancer agent used as the free base. The heterodimeric dual-domain compound is isolated in 55% yield. Various tether lengths will complete the collection, providing the opportunity to perform various studies.

MDR reversal assays with the MCF7adrR and CHO taxol resistant cells have been performed with the dual-domain compounds of the present invention. The compounds of the invention are used for the treatment of resistant forms of cancer. They can be added in conjunction with existing anti-cancer drugs, such as adriamycin, taxol, etc. to treat cancers that are not responding to standard levels of drug. For example, the compounds restore the cytotoxicity of the anti-cancer agent adriamycin to resistant human breast cancer cells (MCF7adrR) at low concentration (260 nM, with 2 nM adriamycin). The invention compounds are less toxic and have lower therapeutic indices (0.25, vs 5 for verapamil).

All patents, publications, and commercial materials cited herein are hereby incorporated by reference.

### EXAMPLES

The following examples are given to illustrate various embodiments which have been made with the present invention. It is to be understood that the following examples are not comprehensive or exhaustive of the many types of embodiments which can be prepared in accordance with the present invention.

### Example 1

### Synthesis of Homodimeric MDR Reversal Agents

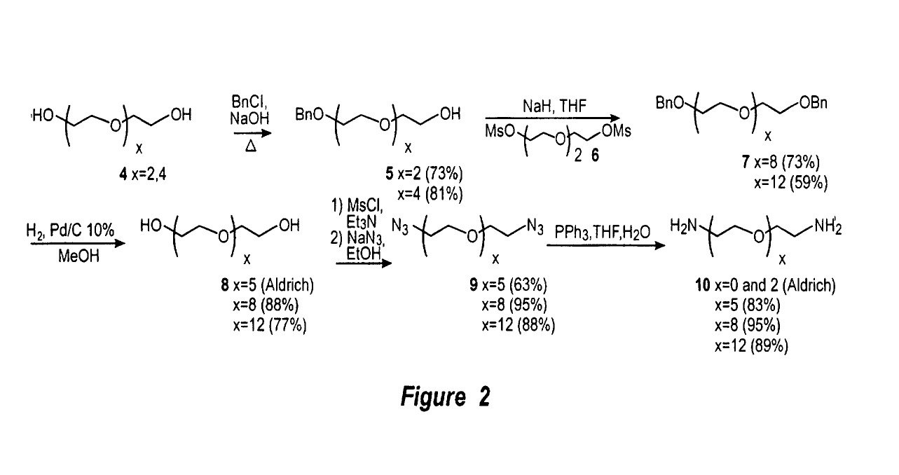

Referring now to FIG. 2, the key to the synthesis of the homodimers was the development of a general, efficient route to synthesize the polyethylene glycol linkers that are not commercially available. Zalipsky, S. Bioconj. Chem. 1995, 6: 150. It was found that mesylates function as an efficient coupling partners. Coudert, G. et al. Syn. Commun. 1986, 16: 19; Keegstra, E. M. D. et al. J. Org. Chem. 1992, 57: 6678. Diols 4 (X=2,) were first monobenzylated using 50% aqueous hydroxide at reflux to give the protected alcohols 5. The step that allowed for reproducible glycol production employed sodium hydride with the alcohol 5, followed by dropwise addition of dimesylate 6 and reflux to provide 7.

All intermediates were characterized by ¹H and ¹³C NMR, and HMRS. Dimesylate 6 was produced from triethylene glycol, mesyl chloride (2.1 equivalents), and triethylamide (2.4 equivalents) in methylene chloride (0.2 M). Sodium bicarbonate work-up and silica gel chromatography were used to isolate the product (97%).

To produce compound 7 with eight ethylene glycols spacers, 95% NaH (0.526 g, 20 mmol) was added to 20 mL THF followed by tri(ethylene glycol) benzylether 5 (x=2) (5.0 g, 20 mmol in 12 mL THF) and the mixture was stirred for 1 hour. Tri(ethylene glycol) dimesylate 6 (3.18 g, 10 nmol in 7.5 mL THF) was added dropwise over 30 min and the solution was refluxed for 20 h. Upon cooling, the solution was diluted with NaHCO₃(sat.) (250 mL) and extracted with CH₂Cl₂. The combined organic layers were dried (MgSO₄), filtered, and concentrated to give a yellow oil. Flash chromatography (100% EtOAc to 10% MeOH/EtOAc) gave the desired product as an oil: 4.51 g (73% yield). Rf=0.34 (10% MeOHIEtOAc); ¹H NMR. (300 MHz, CDCl₃) δ 7.34-7.26 (m, 10H), 4.56 (s, 4H), 3.69-3.61 (m, 36H); ¹³C NMR (75 MHz, CDCl₃) δ 138.4, 128.5, 127.9, 127.8, 73.4, 70.8, 70.7, 69.6; HRMS FAB (M+Na) calc'd for C₃₂H₅₀O₁₀Na 617.3287, found 617.3272.

To prepare compound 7 with twelve ethylene glycol spacers, the same conditions were used as to prepared the eight spacer compound, except that 5 (X=4) was used (59% yield). Rf=0.21 (10% MeOH/EtOAc); ¹H NMR (300 MHz, CDCl₃) δ 7.35-7.27 (m, 10H), 4.57 (s, 4H), 3.69-3.60 (m, 52H; ¹³C NMR (75 MHz, CDCl₃) δ 138.4, 128.5, 127.9, 127.8, 73.4, 70.8, 70.7, 69.6; HRMS FAB (M+Na) calc'd for C₄₀H₆₆O₁₄Na 793.4362, found 793.4374.

After the synthesis of compound 7, hydrogenation at 200 psi with palladium on carbon (10%) in methanol was used to give the diols 8. Dimesylate formation and sodium azide displacement provided diazides 9. Commercially available hexaethylene glycol 8 (x=5) was used for conversion to 9 (x=5). Reduction with triphenylphosphine was used to generate the key diamines 10. Completing the series are 10 (x=0 and 2), which were purchased from commercial suppliers.

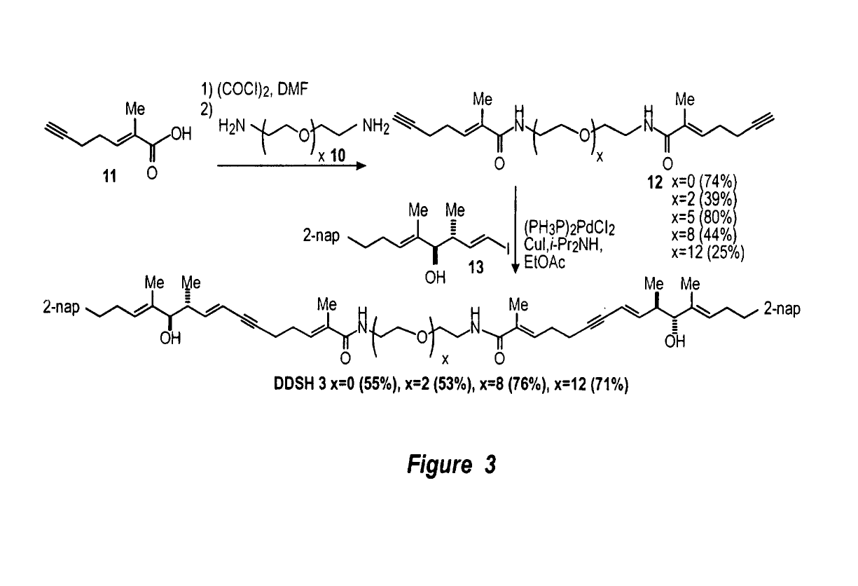

Referring now to FIG. 3, treatment of the diamines 10 (1.2 equivalents) at −40° C. with the known acid (1 equivalent) generated from carboxylic acid 11 led to the formation of the diamides. Difficulties with low yields in some cases (x=8, 12) may be attributed to the very hydroscopic nature of the diamine glycols. Double Sonogashira couplings were then performed with the bis-amide alkynes 12 reacted with the previously reported vinyl iodide 13 (3 equivalents) under palladium chloride, copper iodide catalysis. These optimized conditions include diisopropylamine together with the moderately polar ethyl acetate as solvent and a starting temperature of −20° C. followed by immediate warming to room temperature. The yields for this double process were moderate (50%) for the shorted linked amides 3 (x=O, 2, 5) and good (70%) for the longer products 3 (x=8, 12).

To prepare homodimer 3 of the invention, compound 13 (47 mg, 0.115 mmol) and EtOAc (3.9 mL) were added to a flask containing dialkynylamide 12 (x=0) (11.5 mg, 0.038 mmol). The reaction was cooled to −20° C. and (Ph₃P)₂PdCl₂ (4.1 mg, 0.006 mmol), CuI (3.8 mg, 0.02 mmol), and i-Pr₂NH (0.58 mL, 0.2M) were added. The flask was. removed from the cold bath and allowed to warm to room temperature. The solution was filtered through a silica gel plug using 30% MeOH/EtOAc and concentrated. Purification via radial chromatography (100% EtOAc) gave 18.0 mg (55% yield) of 3 (x=0) as an oil. R_{f}=0.5 (2% MeOH/EtOAc); ¹H NMR (300 MHz, CDCl₃) 67.81-7.67 (m, 6H), 7.60 (s, 2H), 7.47-7.40 (m, 4H), 7.34 (dd, J=8.4, 1.5 Hz, 2H), 6.61 (bs, 2H) 6.38 (t, J=5.7 Hz, 2H), 5.98 (dd, J=15.9, 8.4 Hz, 2H), 5.52 (dd, J=15.9, 0.9 Hz, 2H) 5.45 (t, J=6.6 Hz, 2H), 3.63 (d, J=8.7 Hz, 2H), 3.48-3.46 (m, 4H), 2.87-2.80 (m, 4H), 2.49-2.29 (m, 14H), 1.85 (s, 2H), 1.84 (s, 6H), 1.56 (s, 6H), 0.77 (d, J=6.6 Hz, 6H); ¹³C NMR (75.5 MHz, CDCl₃) δ 170.6, 145.9, 139.6, 135.9, 134.6, 133.7, 132.1, 131.9, 128.3, 128.0, 127.8, 127.6, 127.5, 126.6, 126.1, 125.3, 111.3, 88.4, 81.8, 79.9, 41.5, 40.7, 35.9, 29.6, 28.0, 19.1, 16.9, 13.1, 11.2; HRMS FAB (M+Na) calc'd for C₅₈H₆₈O₄Na 879.5071, found 879.5065. Compounds 3 (X=2,5,8,12) were formed followed the same procedure with yields indicated.

### Example 2

### Synthesis of a Monomeric Control Substrate

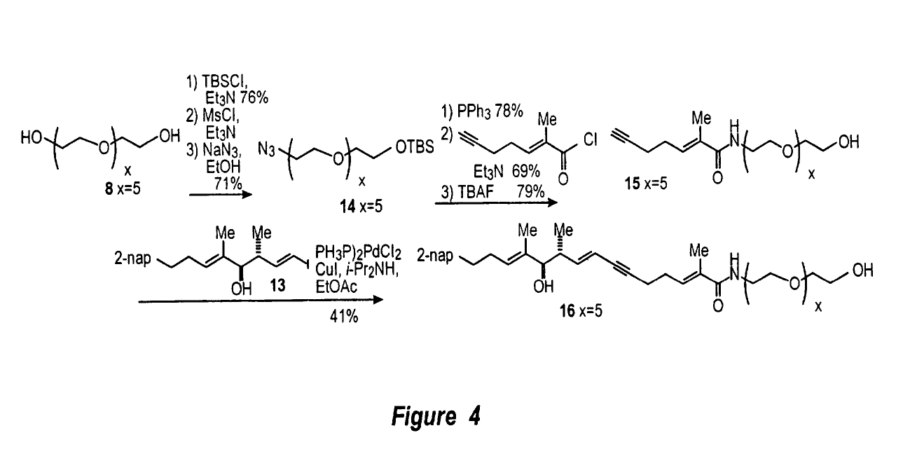

Referring now to FIG. 4, a monomeric ethylene glycol-amide reversal agent was also made for control purposes in MDR and Pgp assays. Monosilyl ether protection, mesylation and azide displacement were uneventful with hexaethylene glvcol 8 (x=5) to give 14. Phosphine reduction, acid chloride coupling, and TBAF deportation generated amide 15. Coupling with 13 then gave the desired control substrate 16.

### Example 3

### Inhibition of ATPase Activity and IAAP Binding by Homodimers and Control Amide 16

Pgp ATPase stimulation activity was determined along with displacement of the prazosin analog, iodoarylazidoprazosin (¹²⁵IAAP). Ambudkar, S. V. Methods Enzymol 1998, 292: 504-514; Dey, S. et al. Methods Enzymol. 1998, 292: 318.

The effect on ATPase activity of Pgp and binding of the IAAP substrate to Pgp are shown in Table 1. The effect of the homodimers of the present invention on ATPase activity and IAAP binding to Pgp are very potent. ATPase stimulation reaches a maximum at low concentration (1 μM) and steadily drops off as the concentration is increased (not shown). In contrast with the other homodimers, the X=5 compound slowly achieves maximum stimulation up to 50 μM and this level is maintained as the concentration increases. The other dimers are similar to the monomeric compounds where maximum stimulation is achieved and rapidly drops off as concentration increases. More surprising are the Pgp displacement of photoaffinity labeled substrate analog by the heterodimers. For example, with the heterodimer where X=12 is shown to bind tightly at 1.7 μM. The other dimers were clearly less effective at ¹²⁵IAAP displacement (10-20 μM).

TABLE 1
Inhibition of IAAP Binding by Homodimers and Control Amide 16

| | | | ATPase^{b} | |
|---|---|---|---|---|
| cmpd | X = | d (Å)^{a} | activity | Ki^{c} |
| 2 | | | 3.20 | 2.7 |
| 16 | | | 0.34 | 7.6 |
| 3 | 0 | 3 | 3.30 | 19.1 |
| 3 | 2 | 11 | 1.90 | 9.7 |
| 3 | 5 | 22 | 1.90 | 15.1 |
| 3 | 8 | 35 | 0.70 | 10.1 |
| 3 | 12 | 50 | 0.00 | 1.7 |

^{a}Approximate distance between amide nitrogens.
^{b}ATP-hydrolysis, fraction of control, values >1 represent stimulation, values <1 represent inhibition.
^{c}K₁ (μM) for inhibition of ¹²⁵IAAP binding to Pgp.

The invention may be embodied in other specific forms without departing from its essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

1. A composition comprising a compound of the formula: 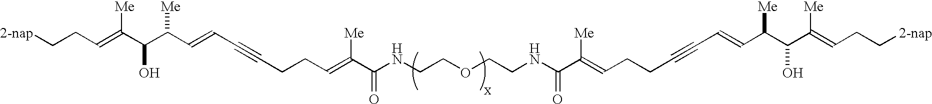
wherein X represents a number of joined ethylene glycol spacers.

2. The composition of claim 1, wherein X is in the range of 0 to about 20.

3. The composition of claim 1, wherein X is in the range of from 2 to about 14.

4. The composition of claim 1, wherein X is about 12.

5. The composition of claim 1, wherein the number of joined ethylene glycol spacers have a length in the range from about 3 Å to about 50 Å.

6. The composition of claim 1, wherein the number of joined ethylene glycol spacers have a length of about 50 Å.

7. A method of reversing multiple drug resistance in a human cell comprising:
administering a composition comprising a compound of the formula: 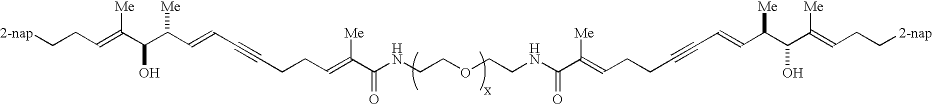
wherein X represents a number of joined ethylene glycol spacers.

8. The method of claim 7, wherein X is in the range of 0 to about 20.

9. The method of claim 7, wherein X is in the range of from 2 to about 14.

10. The method of claim 7, wherein X is about 12.

11. A method of inhibiting substrate binding to Pgp, the method comprising:
contacting a Pgp molecule with a compound of the formula 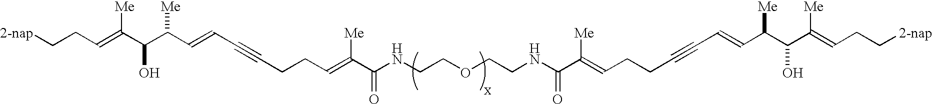
wherein X represents a number of joined ethylene glycol spacers.

12. The method of claim 11, wherein X is in the range of 0 to about 20.

13. The method of claim 11, wherein X is in the range of 2 to about 14

14. The method of claim 11, wherein X is about 12.

15. A method of inhibiting ATPase activity of Pgp comprising:
contacting a Pgp molecule with a compound of the formula 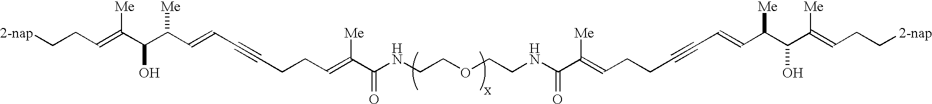
wherein X represents a number of joined ethylene glycol spacers in the range from about 0 to about 20.

16. The method of claim 15, wherein X is in the range of from 2 to about 14

17. The method of claim 15, wherein X is about 12.

18. A method of synthesizing a homodermeric multiple drug resistance reversal agent, the method comprising:
obtaining a first compound represented by the formula 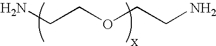
wherein X represents a number of joined ethylene glycol spacers;
reacting the first compound with a second compound represented by the formula 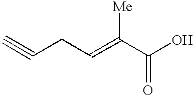
to form a third compound represented by the formula 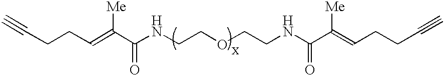
and converting the third compound a fourth compound of the formula

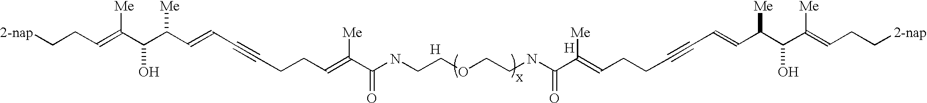

19. The method of claim 18, wherein X is in the range of from 0 to about 20.

20. The method of claim 18, wherein X is in the range from about 2 to about 14.

21. The method of claim 18, wherein X is about 12.

22. The method of claim 18, wherein the first compound is obtained by a method comprising obtaining a fifth compound of the formula 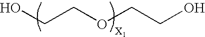
wherein X1 represents 2 or 4 ethylene glycol spacers,
converting the fifth compound to a sixth compound of the formula 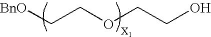
converting the sixth compound to a seventh compound of the formula 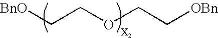
wherein X2 represents 8 or 12 ethylene glycol spacers,
converting the seventh compound to an eighth compound of the formula 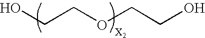
converting the eighth compound to a ninth compound of the formula 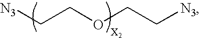
and converting the ninth compound to the first compound.

23. A composition comprising a compound of the formula: 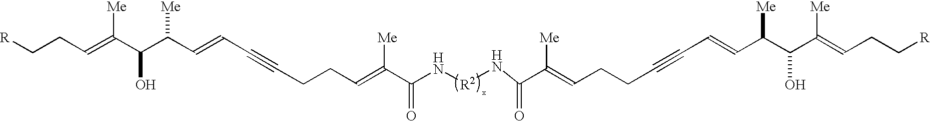
wherein R represents a substituted phenyl group, R² represents joined hydrocarbon spacers, and X represents a number of joined hydrocarbon spacers.

24. The composition of claim 23, wherein the number of joined hydrocarbon spacers have a length in the range from about 3 Å to about 50 Å.

25. The composition of claim 23 wherein the number of joined hydrocarbon spacers have a length of about 50 Å.

26. A method of reversing multiple drug resistance in a human cell comprising contacting the human cell with the composition of claim 23.

27. A method of inhibiting substrate binding to Pgp, the method comprising contacting a Pgp molecule with the composition of claim 23.

28. A method of inhibiting ATPase activity of Pgp comprising contacting a Pgp molecule with the composition of claim 23.

29. A polyvalent MDR reversal agent comprising:
two or more stipiamide-based domains; and
a polyvalent linker for joining the one or more stipiamide-based domains.

30. The polyvalent MDR reversal agent of claim 29, wherein the linker is selected from the group consisting of a bivalent linker, a trivalent linker, a tetravalent linker, and a cyclic hydrocarbon linker.
